(19) United States
(12) Patent Application Publication
(10) Pub. No.: US 2010/0217327 A1
(43) Pub. Date: Aug. 26, 2010

(54) PLATE AND SYSTEM FOR LATERAL TREATMENT OF A FRACTURE OF THE CALCANEUS

(76) Inventors: David W. Vancelette, San Diego, CA (US); Rebecca Hawkins Wahl, Escondido, CA (US); Lew C. Schon, Baltimore, MD (US); Ronald George Litke, Danbury, CT (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/711,427

(22) Filed: Feb. 24, 2010

Related U.S. Application Data
(60) Provisional application No. 61/155,021, filed on Feb. 24, 2009.

(30) Foreign Application Priority Data
May 15, 2009 (FR) 0935233

(51) Int. Cl. A61B 17/80; A61B 17/88

(52) U.S. Cl. 606/281; 606/286

(57) ABSTRACT

A plate includes a substantially rectilinear posterior tab and an anterior tab. The anterior tab includes a substantially rectilinear rear longitudinal part connected rigidly to the posterior tab such that they form between them a first angle of between about 90° and about 110° and a substantially rectilinear front longitudinal part which is inclined with respect to the substantially rectilinear rear longitudinal part such that they form between them a second angle which is between about 230° and about 250°. The second angle is at the same angular orientation as the first angle. The plate is thus shaped appropriately in relation to the calcaneus and can be placed in position in a minimally invasive manner.

### CROSS-REFERENCE TO RELATED APPLICATION

This patent application claims the benefit of U.S. Provisional Patent Application No. 61/155,021, filed on Feb. 24, 2009 and also claims priority to French Patent Application No. 0935233, filed on May 15, 2009. The entire content of these applications are hereby incorporated by reference.

### TECHNICAL FIELD

The present invention relates generally to the field of calcaneus fracture fixation. In particular, the present invention relates to a plate for lateral treatment of a fracture of a human calcaneus, a treatment system using such a plate and a surgical method using the plate.

### BACKGROUND

The calcaneus, sometimes called the heel bone, is the largest bone of the human foot. A fracture of the calcaneus generally includes several fracture lines separating the calcaneus into several bone fragments, especially in the area of its articulation with the talus.

At present, one of the treatments for such a fracture involves a plate of considerable dimensions being placed against the lateral face of the calcaneus and fixed thereto, the plate extending both in an antero-posterior direction and also in a vertical direction in order to cover substantially the whole of the afore-mentioned lateral face. Examples of this type of plate are given in US 2008/0021452; U.S. Pat. No. 6,235,032, U.S. Pat. No. 6,348,052; U.S. Pat. No. 6,123,709 and US 2007/0162020. Given their substantial size, these plates require large incisions, generally L-shaped incisions, to be made in the soft tissue parts covering the lateral face of the patient's calcaneus. Under these conditions, these soft tissue parts containing vessels and nerves are generally irreversibly damaged, not to mention the risk of their becoming infected and of other problems associated with scarring. Moreover, without detaching an even larger area of these soft tissue parts transversely with respect to the area of positioning of the plate, the articulation between the calcaneus and the talus is not directly accessible through the incisions so the surgeon cannot be certain that treatment of the fracture by the plate is satisfactory with regard to this articulation.

The aim of the present invention is to make available a plate for lateral treatment of the calcaneus, which plate has a more advantageous configuration and can be fitted in place in a less invasive manner.

### SUMMARY

The present invention relates to a plate for lateral treatment of a fracture of a human calcaneus, including a substantially rectilinear posterior tab and an anterior tab which includes, firstly, a substantially rectilinear rear longitudinal part connected rigidly to the posterior tab such that they form between them a first angle of between about 90° and about 110°, and, secondly, a substantially rectilinear front longitudinal part which is inclined with respect to the rear part such that they form between them a second angle which, with the same angular orientation as the first angle, is between about 230° and about 250°.

The invention also relates to a plate for lateral treatment of a fracture of a human calcaneus, including a substantially rectilinear posterior tab and a substantially rectilinear anterior tab which is connected rigidly to the posterior tab such that they form between them an angle of between about 110° and about 130°.

The invention further relates to a surgical method for lateral treatment of a fracture of a calcaneus, in which method: one of the treatment plates defined above is provided, an incision is made in the soft tissue parts of the patient in the area of the thalamic facet of the calcaneus until the thalamic facet can be observed, the plate is inserted into the incision and placed against the upper half of the lateral face of the calcaneus in such a way that its posterior and anterior tabs extend successively from the tuberosity to the greater process of the calcaneus, passing across the calcaneus substantially opposite from its lesser process in the medio-lateral direction, and the plate is fixed to the calcaneus.

The invention is based on the concept of permitting minimally invasive treatment of the calcaneus. To this end, it will be noted that the most commonly occurring fracture lines of the calcaneus are, on the one hand, a first vertical line extending from the anterior part of the upper face of the calcaneus, generally from its thalamic facet, that is to say its main articular facet with the talus, to the lower face of the calcaneus, generally as far as the surface lying between the tuberosities of this face, and, on the other hand, a second antero-posterior line which starts from the posterior face of the calcaneus, generally from the upper part of the posterior tuberosity, and joins up with the first line. By virtue of its posterior and anterior tabs, which are designed to extend along the lateral face of the calcaneus, from the tuberosity thereof to the greater process, passing the level of its lesser process, the plate according to the invention is able to transversely straddle the two aforementioned fracture lines, with the anterior tab permitting reduction mainly of the first line, while the posterior tab permits reduction mainly of the second line. The shape of the plate, which is generally bent at the joint between the posterior and anterior tabs, thus proves particularly efficient for covering the upper half of the lateral face of the calcaneus and, upon fixation to the calcaneus by any suitable means, for capturing the bone fragments separated by the most common fracture lines. Moreover, fitting this plate causes less trauma to the soft tissue parts of the patient: it requires only a small, substantially rectilinear incision just below the talus in the area of the thalamic facet, then insertion of the plate into this incision downwards along the lateral face of the calcaneus. This minimally invasive surgical approach allows the thalamic facet to be observed directly, which facilitates restoration thereof during treatment of the fracture of the calcaneus. Thus, treatment by means of the plate according to the invention restores the anatomical dimensions of the calcaneus with very easy implantation and good post-operative results.

According to other advantageous features of the plate according to the invention, taken either separately or in all the technically possible combinations: the plate is composed exclusively of its posterior tab and of its anterior tab; the posterior tab and anterior tab have a substantially constant width in their longitudinal direction except in the area of the rear end of the posterior tab and of the front end of the front part of the anterior tab; the rear end of the posterior tab and the front end of the front part of the anterior tab each have a curved peripheral profile and are less thick than the rest of the plate; the rear end of the posterior tab and the front end of the front part of the anterior tab are each bevelled, with their thickness decreasing from the rest of the plate to their free edge with the curved profile; one and/or both of the posterior tab and anterior tab is/are provided with at least one transverse groove for folding the tab on itself; and the face of the posterior tab and anterior tab directed towards the calcaneus is concave in places.

The invention also relates to a system for lateral treatment of a fracture of a human calcaneus, including: a treatment plate as defined above, at least one locking screw designed to be introduced into a through-hole of the plate and to fix the plate mechanically to the calcaneus, and at least one positioning screw designed to be introduced into a through-hole of the plate and to adjust and enforce the positioning of at least one fragment of the calcaneus with respect to the plate.

Advantageously, the treatment system additionally includes instrumentation for percutaneously targeting at least one of the through-holes of the plate and for guiding the one or more locking screws and/or the one or more positioning screws into this hole.

In one embodiment, the present invention is a plate for lateral treatment of a fracture of a human calcaneus. The plate includes a substantially rectilinear posterior tab and an anterior tab including a substantially rectilinear rear longitudinal part rigidly connected to the posterior tab and a substantially rectilinear front longitudinal part. The anterior tab and the posterior tab form between them a first angle of between about 90° and about 110°. The substantially rectilinear front longitudinal part is inclined with respect to the substantially rectilinear rear longitudinal part such that the substantially rectilinear front longitudinal part and the substantially rectilinear rear longitudinal part form between them a second angle of between about 230° and about 250°, where the second angle has the same angular orientation as the first angle. In one embodiment, the anterior tab and the posterior tab form between them an angle of between about 110° and about 130°.

In another embodiment, the present invention is a system for lateral treatment of a fracture of a human calcaneus including the plate described above, at least one locking screw designed to be introduced into a through-hole of the plate and to mechanically fix the plate to the calcaneus and at least one positioning screw designed to be introduced into a through-hole of the plate and to adjust and enforce positioning of at least one fragment of the calcaneus with respect to the plate.

In yet another embodiment, the present invention is a surgical method for lateral treatment of a fracture of a calcaneus of a human patient The method includes providing a plate as described above, making an incision in soft tissue parts of the patient in an area of a thalamic facet of the calcaneus until the thalamic facet can be observed, inserting the plate into the incision; placing the plate against an upper half of a lateral face of the calcaneus in such a way that the posterior and anterior tabs extend successively from a tuberosity to a greater process of the calcaneus, passing across the calcaneus substantially opposite from a lesser process of the calcaneus in a medio-lateral direction and fixing the plate to the calcaneus.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The invention will be better understood from the following description which is given solely by way of example and with reference to the drawings, in which:

FIG. 1 is an elevation view of osseous components of a human foot, of which a calcaneus is treated by a plate according to the present invention;

FIG. 2 is a perspective view of a part of the foot shown in FIG. 1;

FIG. 3 is an elevation view of the plate shown in FIGS. 1 and 2;

FIG. 4 is an elevation view in the direction of arrow IV in FIG. 3;

FIG. 5 is a view similar to FIG. 2, showing another embodiment of a plate according to the present invention;

FIG. 6 is an elevation view of the plate shown in FIG. 5; and

FIG. 7 is an elevation view in the direction of arrow VII in FIG. 6.

### DETAILED DESCRIPTION

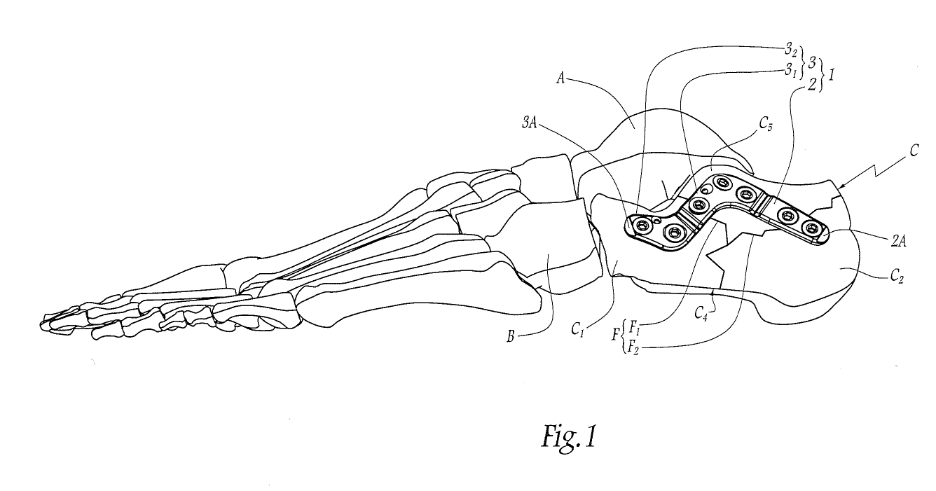

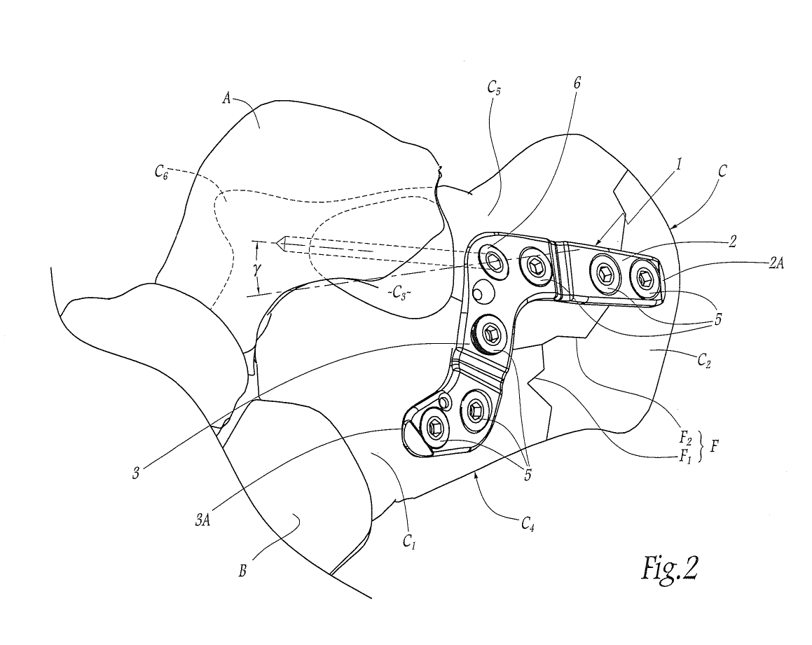

FIGS. 1 to 4 are views showing a plate 1 for lateral treatment of a fracture F of the calcaneus C. The calcaneus C and other osseous components of the foot of a patient are shown schematically in FIGS. 1 and 2. For example, the talus is labeled with reference letter A and the cuboid bone is labeled with reference letter B.

As is shown in FIGS. 1 and 2, the fracture F of the calcaneus C generally includes two fracture lines F₁ and F₂, which correspond to the fracture lines most commonly found in patients treated for a fracture of the calcaneus C. The fracture line F₁ is a generally vertical line which is situated in the antero-posterior direction between the greater process C₁ and the tuberosity C₂ of the calcaneus C and extends from the thalamic facet C₃ (FIG. 2) of the upper face of the calcaneus C, that is to say its main articular facet with the talus A, to the surface C₄ between the tuberosities of the lower face of the calcaneus C. The fracture line F₂ runs in what is generally an antero-posterior direction from the posterior face of the tuberosity C₂ to the posterior root of the greater process C₁ where it joins up with the fracture line F₁.

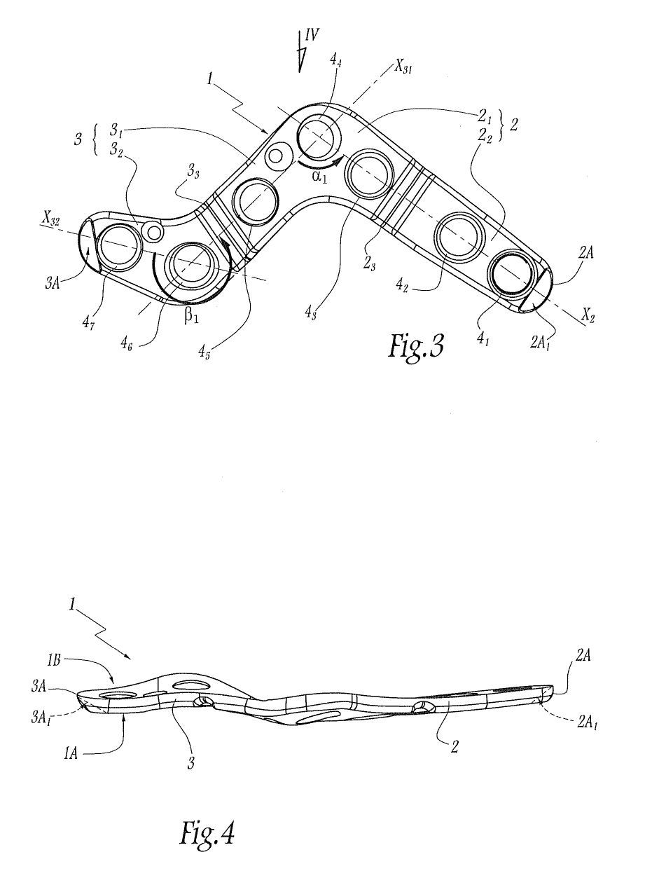

As is shown in FIGS. 3 and 4, the plate 1 includes a posterior tab 2 and an anterior tab 3. The term “tab” is used to designate a plate part which has a smaller thickness than the two other dimensions of the plate 1 and which, when observed in the direction of its thickness as in FIG. 3, has an elongate and optionally bent shape. The smallest dimension corresponds to the width of the plate 1.

Thus, for the plate 1 shown in FIGS. 1-4, the posterior tab 2 is rectilinear in its longitudinal direction, being centered on an axis X₂, and has a substantially constant width along its entire length except in the area of its rear end 2A, as is explained in detail below. The anterior tab 3 is bent and includes a rectilinear rear longitudinal part 3₁ centered on an axis X₃₁ and a rectilinear front longitudinal part 3₂ centered on an axis X₃₂. The width of the tab 3 is constant in its longitudinal direction, that is to say along the axes X₃₁ and X₃₂, except in the area of the front end 3A of the front longitudinal part 3₂, as is explained in detail below.

The posterior tab 2 and anterior tab 3 are rigidly connected to each other by being produced as one piece, although it is also alternatively possible for them to be joined together by any suitable means, for example by adhesive bonding or welding. More precisely, as can be clearly seen in FIG. 3, the rear longitudinal part 3₁ of the anterior tab 3, more specifically the rear end of the rear longitudinal part 3₁, is connected rigidly to a front part 2₁ of the posterior tab 2. Specifically, the rear longitudinal part 3₁ of the anterior tab 3 is connected to the front end of the front part 2₁ to form an angled connection. The axes X₃₁ and X₂ thus define between them an angle₁, which is not zero and is less than about 180°. In one embodiment, the angle₁ is strictly less than 180°.

Likewise, the rear longitudinal part 3₁ and front longitudinal part 3₂ of the anterior tab 3, which are joined rigidly via their front and rear ends respectively, together form an angle₁ between the axes X₃₂ and X₃₁. On the basis of the angular orientation of the angle₁, the aforementioned angle₁ is greater than about 180° and less than about 360°, giving the plate 1 a generally S-shape. In one embodiment, the angle₁ is strictly greater than 180° and strictly less than 360°. In other words, the front part 2₁ of the posterior tab 2 and the front longitudinal part 3₂ of the anterior tab 3 extend respectively from the two opposite longitudinal ends of the rear longitudinal part 3₁ of the anterior tab 3.

Exemplary dimension specifications of the plate 1 include: the angle₁ is between about 90° and about 110°, the angle₁ is between about 230° and about 250°, the posterior tab 2 has a length, along its axis X₂, of between about 32 mm and about 46 mm, the rear part 3₁ of the anterior tab 3 has a length of between about 22 mm and about 28 mm along its axis X₃₁, the front part 3₂ has a length substantially equal to about 20 mm along its axis X₃₂, and the width of the tabs 2 and 3 is between about 7 mm and about 12 mm.

The rear end 2A of the posterior tab 2 and the front end 3A of the anterior tab 3 constitute free rear and front ends 2A and 3A, respectively, of the plate 1 and for this reason, have particular configurations. When the plate 1 is observed in the direction of its thickness as in FIG. 3, each of these ends 2A and 3A has a curved peripheral profile. In the embodiment shown, the profile is rounded such that, without any angular discontinuity, the two opposite longitudinal edges either of the rear part 2₂ of the posterior tab 2 or of the front longitudinal part 3₂ of the anterior tab 3 are connected. Moreover, as is indicated by the broken lines in FIG. 4 and as can be clearly seen in FIGS. 1 and 2, the ends 2A and 3A have a thickness less than that of the rest of the plate 1. Each of the ends 2A and 3A is in fact bevelled in such a way that its thickness decreases progressively from the rest of the plate 1 towards its free edge with the curved profile. The front face 1A of the plate 1 is designed to be directed away from the calcaneus C during use such that the ends 2A and 3A delimit respective surfaces 2A₁ and 3A₁ which are plane and are also inclined with respect to the rest of the front face 1A.

Also on its front face 1A, the plate 1 has two transverse grooves formed in it, namely a groove 2₃ which extends substantially perpendicularly with respect to the axis X₂ and which is situated between the front part 2₁ and rear part 2₂ of the posterior tab 2, and a groove 3₃ which extends substantially perpendicularly with respect to the axis X₃₁ and which is situated in the median zone of the rear part 3₁ of the anterior tab 3. The groove 2₃ forms a line for folding the posterior tab 2 on itself, making it possible to slightly tilt the front part 2₁ and rear part 2₂ relative to each other about a geometrical fold axis which is perpendicular to the axis X₂ and is situated at the bottom of the groove 2₃. Likewise, the groove 3₃ forms a line for folding the tab part 3₁ on itself, making it possible to slightly tilt the two halves of this tab part 3₁ relative to each other about a geometrical fold axis which is perpendicular to the axis X₃₁ and is situated at the bottom of the groove 3₃.

Moreover, as can be seen in FIG. 4, the rear face 1B of the plate 1 designed to be directed towards the calcaneus C during use is not absolutely plane and instead is concave in places, so as to better match the rear face 1B to the lateral face of the calcaneus C against which the plate 1 is designed to be placed, as is explained below.

The plate 1, when in use, is associated with locking screws 5 and positioning screws 6 (shown in FIG. 2) which are received in through-holes (shown in FIG. 3) extending through the faces 1A and 1B of the plate 1. In the embodiment of FIG. 3 shows seven through-holes which are labeled as follows: 4₁ to 4₃ for the three holes, distributed from the rear to the front, exclusively in the posterior tab 2; 4₄ for the hole situated in the joining zone between the tabs 2 and 3; 4₅ for the hole situated exclusively in the rear part 3₁ of the anterior tab 3; 4₆ for the hole situated in the bent joining zone between the parts 3₁ and 3₂ of the anterior tab 3; and 4₇ for the hole situated exclusively in the front part 3₁ of the anterior tab 3. Details on the holes 4₁-4₇ and the screws 5 and 6 will be given hereinafter in the context of an example of the treatment of the fracture F of the calcaneus C using the plate 1.

In a first operating step, a surgeon makes an incision in the soft tissue parts of the patient, laterally exposing the articulation between the calcaneus C and the talus A. The surgeon thus makes a substantially rectilinear antero-posterior incision in the area of the thalamic facet C₃ of the calcaneus C, the extent of the incision being relatively short. In one embodiment, the incision is a few centimeters long. The incision is made to a depth that allows the surgeon to directly observe the thalamic facet C₃. The surgeon is then able to visually determine the damage to the thalamic facet C₃ due to the fracture F, in particular by examining the upper end of the fracture line F₁.

In a second operating step, the surgeon maneuvers the plate 1 which, for example, is initially in the configuration shown in FIGS. 3 to 4. If appropriate, depending on data relating to the degree of convexity of the lateral face of the calcaneus C, which data can be made available to the surgeon by pre-operative X-rays or by palpation of the bones, the surgeon adjusts the concavity of the rear face 1B of the plate 1 by bending and/or by folding one and/or both of the grooves 2₃ and 3₃.

The surgeon then inserts the ends 2A and 3A of the plate 1 into the incision. The inclined surfaces 2A₁ and 3A₁ of the ends 2A and 3A facilitate the insertion and movement of the ends 2A and 3A in the patient's foot, more specifically along the lateral face of the calcaneus C from the lateral margin of the thalamic facet C₃ in the direction of the lower face of the calcaneus.

The surgeon continues inserting the plate 1 into the incision until the plate 1 is placed against the upper half of the lateral face of the calcaneus C, as shown in FIGS. 1 and 2. In this configuration, the posterior tab 2 extends from its rear end 2A to its front end between the tuberosity C₂ and an upper region C₅ of the lateral face of the calcaneus C situated in the medio-lateral direction across the bone from the lesser process C₆ (shown in FIG. 2) present on the medial face of the calcaneus C. The posterior tab 2 thus transversely straddles the fracture line F₂. The anterior tab 3 extends from its rear end to its front end 3A between the upper region C₅ and the greater process C₁ of the calcaneus C in such a way that its rear part 3₁ transversely straddles the fracture line F₁.

It will be appreciated in practice that the ability of the plate 1 to straddle the lines F₁ and F₂ of the fracture F and occupy only the upper half of the lateral face of the calcaneus C is directly linked to its bent shape. In particular, the geometric specifications of the plate 1 are predetermined in such a way that the rear part 3₁ and the front part 3₂ of the anterior tab 3 are parallel to the segments which, in anatomy, define the angle of Gissane. The front end 3A is situated just behind the midpoint of the segment connecting the upper and lower boundaries of the articular facet of the calcaneus C with the cuboid bone B. The posterior tab 2, substantially at its middle, intersects the segment which on the lateral face of the tuberosity C₂ connects the upper surface of the latter and its lower crest, commonly called the “fibular crest”.

In a third operating step, the plate 1 is fixed to the calcaneus C using the screws 5 and 6. More precisely, the locking screws 5 allow the plate 1 to be fixedly locked to the calcaneus C. For this purpose, by way of example, the head of each locking screw 5 is threaded in such a way that after the threaded shank of the locking screw 5 has been introduced through one of the holes 4₁ to 4₇ and screwed into the osseous substance of the calcaneus C, screwing the head of the locking screw 5 in a complementary thread of the respective hole firmly fixes the plate 1 to the calcaneus C. The positioning screws 6 make it possible to adjust and enforce the positioning of the calcaneus C with respect to the plate 1. For example, the head of each positioning screw 6 has a substantially hemispherical lateral surface such that after the threaded shank of the positioning screw 6 has been introduced through one of the holes 4₁ to 4₇ and the shank has been screwed into the osseous substance of one of the fragments of the fractured calcaneus C, the relative positioning between the plate 1 and the calcaneal fragment is such that the bearing between the head of the positioning screw 6 and the wall delimiting the respective hole offsets are transverse with respect to the central axis of the hole, making it possible to move such calcaneal fragments together in order to reduce the fracture F. Moreover, each positioning screw 6 can advantageously cooperate with the hole receiving it and form an angle with the central axis of the hole that is not zero and that is freely chosen by the surgeon. In one embodiment, the angle is about 15°.

As the incision is to be as small as possible, the surgeon does not have direct access to all of the holes 4₁ to 4₇ in order to engage the screws 5 and 6 therein. Under these conditions, all or some of the screws 5 and 6 are advantageously introduced percutaneously with the aid of specific instrumentation with which it is possible to target the holes 4₁ to 4₇ from outside the soft skin tissue and guide the screws 5 and 6 into the holes through the soft tissue parts.

In the embodiments shown in FIGS. 1 and 4, all of the holes 4₁ to 4₇ are threaded except for hole 4₄ whose wall is smooth. The threaded holes receive a locking screw 5 whereas the smooth hole 4₄ receives a positioning screw 6. The positioning screw 6 is made sufficiently long to pass through the calcaneus C from the lateral face to its lesser process C₆, and if appropriate, to pass through the fracture line F₁ in order to effectively reduce the fracture line F₁. If necessary, as has been explained above, the positioning screw 6 is screwed through the calcaneus C at an inclination with respect to a central axis of the hole 4₄, as is indicated by the angle in FIG. 2, such that the shank of the positioning screw 6 reaches the central region of the lesser process C₆.

Because locking screws 5 and positioning screws 6 of this kind and their respective uses are known, they will not be described here in any more detail.

It will be noted that in practice the fold grooves 2₃ and 3₃ in the plate 1 are distinctly delimited from the holes 4₁ to 4₇ so as not to affect the cooperation between the holes and the heads of the screws 5 and 6.

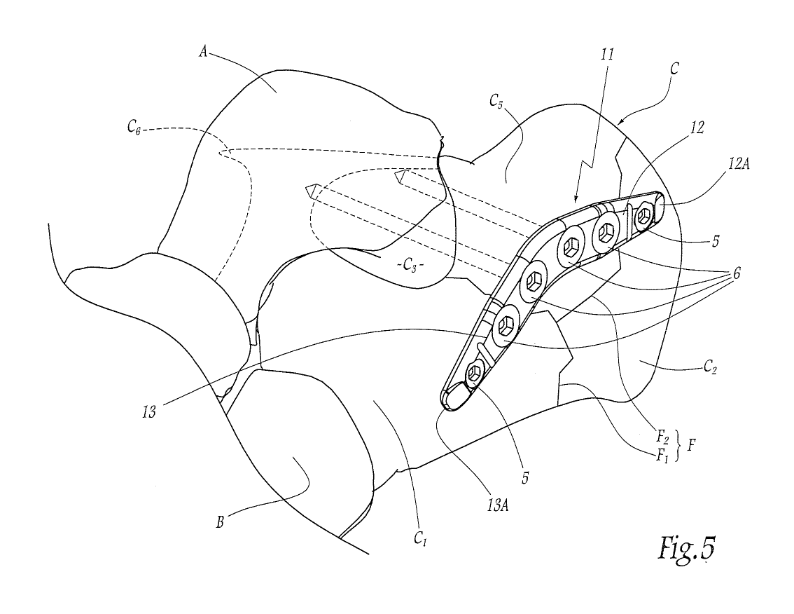

FIGS. 5 to 7 show a plate 11 which is analogous in function to the plate 1 (shown in FIGS. 1-4). In terms of structure, the plate 11 includes, similar to the plate 1, a rectilinear posterior tab 12 centered on a longitudinal axis X₁₂ and an anterior tab 13 which, unlike the tab 3 of the plate 1, is not bent, but is instead rectilinear along its entire length, being centered on an axis X₁₃. In other words, a rear part 13₁ and front part 13₂ of the anterior tab 13 extend in the rectilinear continuation of each other along the axis X₁₃.

As with the plate 1, the rear part 13₁ is rigidly connected at its rear end to the front part 12₁ of the posterior tab 12 in a bent shape such that they form between them an angle₁₁ defined between the axes X₁₃ and X₁₂.

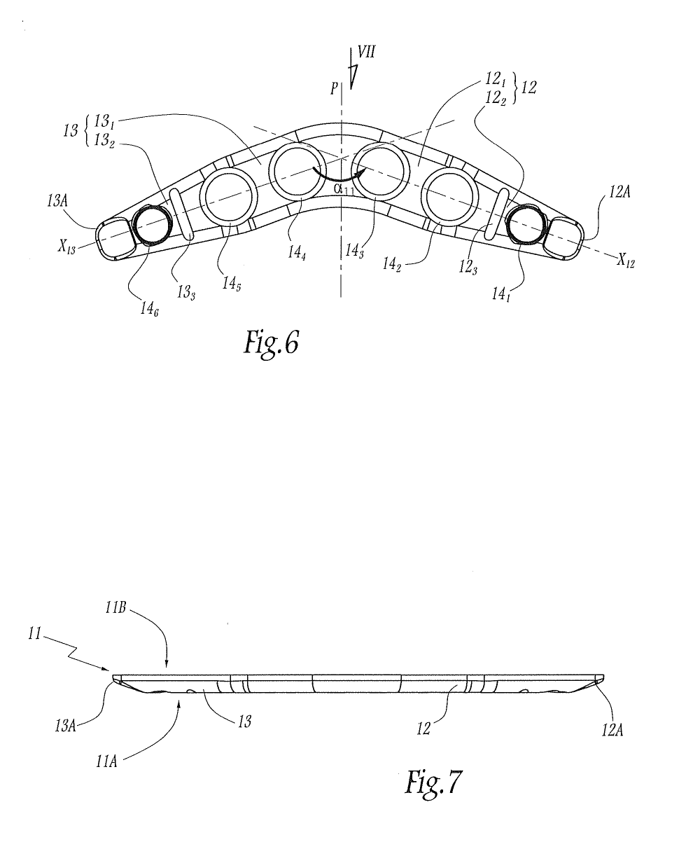

Again in the same way as with the plate 1, the free ends of the plate 11 include, respectively, a rear end 12A of a rear part 12₂ of the posterior tab 12 and a front end 13A of the front part 13₂ of the anterior tab 13. In the same way as for the ends 2A and 3A of the plate 1, and for the same reasons, the ends 12A and 13A have a curved peripheral profile and are bevelled, as can be clearly seen in FIGS. 6 and 7.

Again analogously to the plate 1, the plate 11 is provided with a groove 12₃ located substantially between the front part 12₁ and rear part 12₂ of the posterior tab 12 for folding the posterior tab 12 on itself and with a groove 13₃ located substantially between the rear part 13₁ and front part 13₂ of the anterior tab 13 for folding the anterior tab 13 on itself.

The plate 11 is also provided with six through-holes 14₁ to 14₆ which are analogous in function to the holes 4₁ to 4₇ of the plate 1.

Moreover, as can be clearly seen in FIG. 7, the rear face 11B of the plate 11 is designed to be directed towards the calcaneus C and is substantially planar, unlike the rear rear 1B of the plate 1, which is concave in places.

The treatment of the fracture F by the plate 11 is analogous to that of the plate 1. Thus, after the soft tissue parts of the patient have been incised in the area of the thalamic facet C₃ of the calcaneus C, the plate 11 is inserted into the incision that has been made by first engaging the beveled ends 12A and 13A until the plate 11 is placed against the upper half of the lateral face of the calcaneus C, as is shown in FIG. 5. In this configuration, the posterior tab 12 extends from the rear to the front between the tuberosity C₂ and the region C₅ substantially opposite the lesser process C₆ so as to straddle the fracture line F₂, whereas the anterior tab 13 extends from the rear part 12₂ to the front part 12₁ from the aforementioned region C₅ to the greater process Cᵢ of the calcaneus C so as to straddle the fracture line F₁. The design of the plate 11, which is by and large like that of a boomerang, is predetermined such that the tabs 12 and 13 extend parallel to the segments which between them define an angle complementing Bohler's anatomical angle. By way of example of suitable numerical specifications, the angle₁₁ is between about 110° and about 130° while each of the tabs 12 and 13 has a length of between about 35 mm and about 45 mm with a width of between about 5 mm and about 7 mm.

After the plate 11 has been placed in position, the plate 11 is fixed to the calcaneus C by screws 15 and 16 which are analogous, respectively, to the locking screws 5 and positioning screws 6 (FIG. 2) used with the plate 1. In the embodiment shown in FIG. 5, two locking screws 15 are used and are received respectively in a rear end hole 14₁ and a front end hole 14₆, which holes 14₁ and 14₆ are internally threaded for this purpose, as can be seen in FIG. 6. Four positioning screws 16 are used and are received in the other holes 14₂ to 14₅ which, for this purpose, have a smooth wall. As shown by the broken lines in FIG. 5, the positioning screws 16 engaging the holes 14₃ and 14₄ situated at the joining bend between the tabs 12 and 13 are screwed through the calcaneus C in the direction of the lesser process C₆ in the same way as the positioning screw 6 received in the hole 4₄ of the plate 1.

As indicated by the broken lines in FIG. 5, the tabs 12 and 13 are symmetrical to each other in relation to a plane P which contains the bisecting line of the angle₁₁ and is perpendicular to the plane containing the axes X₁₂ and X₁₃. In this way, the plate 11 can be used equally well to treat a right-hand calcaneus or a left-hand calcaneus. This is in contrast to the plate 1 which is designed to treat a right-hand calcaneus and which, in order to treat a left-hand calcaneus, must be projected symmetrically with respect to a sagittal plane of the patient.

Various set-ups and alternatives to the plates 1 and 11 described above are also conceivable. For example, the plates 1 and 11 can be made of any material suitable for treating the fracture F of the calcaneus C. Exemplary materials suitable for forming plates 1 and 11 include, but are not limited to: a polymer material, a metal alloy, ceramic, pyrolytic carbon, or any other biocompatible material.

In light of the human anatomy, the thickness of the plates 1 and 11 is between about 1 mm and about 3.5 mm.

While the posterior tab 2, 12 and anterior tab 3, 13 are rigidly connected to each other in the area of their front and rear ends, respectively, this rigid connection can more generally be formed between the front part 2₁, 12₁ of the posterior tab 2, 12 and the rear part 3₁, 13₁ of the anterior tab 3, 13. In this case, when the anterior tab 3, 13 is additionally bent like the anterior tab 3 of the plate 1, the plate 1 has the overall shape of the number “4”.

The number, size and configuration of the through-holes of the plates 1 and 11 are not limited to those depicted in the figures. In particular, instead of the holes being distributed along the tabs 2, 12 and 3, 13 in a single row, two or even more rows of holes can be provided, particularly in the front part 2₁, 12₁ of the posterior tab 2, 12 and in the rear part 3₁, 13₁ of the anterior tab 3, 13.

Similarly, the number of locking screws 5, 15 and the number of positioning screws 6, 16 can be modified, it being noted that for reasons of mechanical strength, one solution is to provide at least one locking screw 5, 15 in the hole nearest to each free end 2A, 3A and 12A, 13A of the plate 1, 11. Moreover, the choice of fitting a locking screw 5, 15 instead of a positioning screw 6, 16 can be left to the surgeon during the actual surgical intervention, provided that each of the holes 4₁ to 4₇, 14₁ to 14₆ is adapted equally to receive either of the two screw types.

In addition to the locking screws 5, 15 and positioning screws 6, 16, the plate 1, 11 can be provided with other mechanical fixing elements, such as additional screws for fixing the plate 1, 11 in a manner offset with respect to osseous structures adjacent to the calcaneus C, or such as elements for blocking the locking screws 5, 15 and positioning screws 6, 16 relative to the plate 1, 11.

As has been explained above, the plates 1, 11 are particularly effective at reducing the fracture lines F₁ and F₂. In practice, the plates 1, 11 effectively treat other fracture lines of the calcaneus C, or, more generally, other forms of bone damage of the calcaneus C, for example in the case of osteotomy of part of this bone.

Although the plates 1, 11 are discussed as being placed against the lateral face of the calcaneus C in a minimally invasive manner, the plates 1, 11 can optionally be fitted in place by a traditional surgical access route, that is to say a significantly more invasive one, by making a wide incision in the soft tissue parts covering the lateral face of the calcaneus C.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

The following is claimed:

1. A plate for lateral treatment of a fracture of a human calcaneus, the plate comprising:
a substantially rectilinear posterior tab; and
an anterior tab including a substantially rectilinear rear longitudinal part rigidly connected to the posterior tab such that the anterior tab and the posterior tab form between them a first angle of between about 90° and about 110° and a substantially rectilinear front longitudinal part inclined with respect to the substantially rectilinear rear longitudinal part such that the substantially rectilinear front longitudinal part and the substantially rectilinear rear longitudinal part form between them a second angle of between about 230° and about 250°, wherein the second angle has the same angular orientation as the first angle.

2. The plate of claim 1, consisting essentially of the posterior tab and the anterior tab.

3. The plate of claim 1, wherein the posterior tab and the anterior tab have a substantially constant width in a longitudinal direction except in an area of a rear end of the posterior tab and in an area of a front end of the substantially rectilinear front longitudinal part of the anterior tab.

4. The plate of claim 3, wherein each of the rear end of the posterior tab and the front end of the substantially rectilinear front longitudinal part of the anterior tab has a curved peripheral profile and has a thickness that is less than a thickness of a remainder of the plate.

5. The plate of claim 3, wherein each of the rear end of the posterior tab and the front end of the substantially rectilinear front longitudinal part of the anterior tab are beveled with thicknesses of the rear end of the posterior tab and the front end of the substantially rectilinear front longitudinal part of the anterior tab decreasing from a remainder of the plate to a free edge with a curved profile.

6. The plate of claim 1, wherein at least one of the posterior tab and the anterior tab is provided with at least one transverse groove for folding the tab on itself.

7. The plate of claim 1, wherein a face of the posterior tab and a face of the anterior tab directed towards the human calcaneus is at least partially concave.

8. A plate for lateral treatment of a fracture of a human calcaneus comprising:
a substantially rectilinear posterior tab; and
a substantially rectilinear anterior tab rigidly connected to the posterior tab such that the substantially rectilinear posterior tab and the substantially rectilinear anterior tab form between them an angle of between about 110° and about 130°.

9. The plate of claim 8, wherein the posterior tab and the anterior tab have a substantially constant width in a longitudinal direction except in an area of a rear end of the posterior tab and in an area of a front end of the substantially rectilinear front longitudinal part of the anterior tab.

10. The plate of claim 9, wherein each of the rear end of the posterior tab and the front end of the substantially rectilinear front longitudinal part of the anterior tab has a curved peripheral profile and has a thickness that is less than a thickness of a remainder of the plate.

11. The plate of claim 9, wherein each of the rear end of the posterior tab and the front end of the substantially rectilinear front longitudinal part of the anterior tab are beveled with thicknesses of the rear end of the posterior tab and the front end of the substantially rectilinear front longitudinal part of the anterior tab decreasing from a remainder of the plate to a free edge with a curved profile.

12. The plate of claim 8, wherein at least one of the posterior tab and the anterior tab is provided with at least one transverse groove for folding the tab on itself.

13. The plate of claim 8, wherein a face of the posterior tab and a face of the anterior tab directed towards the human calcaneus is at least partially concave.

14. A system for lateral treatment of a fracture of a human calcaneus comprising:
a treatment plate including a substantially rectilinear posterior tab and an anterior tab including a substantially rectilinear rear longitudinal part rigidly connected to the posterior tab such that the anterior tab and the posterior tab form between them a first angle of between about 90° and about 110° and a substantially rectilinear front longitudinal part inclined with respect to the substantially rectilinear rear longitudinal part such that the substantially rectilinear front longitudinal part and the substantially rectilinear rear longitudinal part form between them a second angle of between about 230° and about 250°, wherein the second angle has the same angular orientation as the first angle;
at least one locking screw designed to be introduced into a through-hole of the plate and to mechanically fix the plate to the calcaneus, and
at least one positioning screw designed to be introduced into a through-hole of the plate and to adjust and enforce positioning of at least one fragment of the calcaneus with respect to the plate.

15. The system of claim 14, further comprising instrumentation for percutaneously targeting at least one of the through-holes of the plate and for guiding the at least one locking screw and/or the at least one positioning screw into the hole.

16. A system for lateral treatment of a fracture of a human calcaneus comprising:
a plate including a substantially rectilinear posterior tab and a substantially rectilinear anterior tab rigidly connected to the posterior tab such that the rectilinear posterior tab and the rectilinear anterior tab form between them an angle of between about 110° and about 130°;
at least one locking screw designed to be introduced into a through-hole of the plate and to mechanically fix the plate to the calcaneus, and
at least one positioning screw designed to be introduced into a through-hole of the plate and to adjust and enforce positioning of at least one fragment of the calcaneus with respect to the plate.

17. The system of claim 16, further comprising instrumentation for percutaneously targeting at least one of the through-holes of the plate and for guiding the at least one locking screw and/or the at least one positioning screw into the hole.

18. A surgical method for lateral treatment of a fracture of a calcaneus of a human patient, the method comprising:
providing a treatment plate including a substantially rectilinear posterior tab and an anterior tab including a substantially rectilinear rear longitudinal part rigidly connected to the posterior tab such that the anterior tab and the posterior tab form between them a first angle of between about 90° and about 110° and a substantially rectilinear front longitudinal part inclined with respect to the substantially rectilinear rear longitudinal part such that the substantially rectilinear front longitudinal part and the substantially rectilinear rear longitudinal part form between them a second angle of between about 230° and about 250°, wherein the second angle has the same angular orientation as the first angle;
making an incision in soft tissue parts of the patient in an area of a thalamic facet of the calcaneus until the thalamic facet can be observed;
inserting the treatment plate into the incision;
placing the treatment plate against an upper half of a lateral face of the calcaneus in such a way that the posterior and anterior tabs extend successively from a tuberosity to a greater process of the calcaneus, passing across the calcaneus substantially opposite from a lesser process of the calcaneus in a medio-lateral direction; and
fixing the treatment plate to the calcaneus.

19. A surgical method for lateral treatment of a fracture of a calcaneus of a human patient, the method comprising:
providing a plate including a substantially rectilinear posterior tab and a substantially rectilinear anterior tab rigidly connected to the substantially rectilinear posterior tab such that the substantially rectilinear posterior tab and the substantially rectilinear anterior tab form between them an angle of between about 110° and about 130°;
making an incision in soft tissue parts of the patient in an area of a thalamic facet of the calcaneus until the thalamic facet can be observed;
inserting the plate into the incision;
placing the plate against an upper half of a lateral face of the calcaneus in such a way that the posterior and anterior tabs extend successively from a tuberosity to a greater process of the calcaneus, passing across the calcaneus substantially opposite from a lesser process of the calcaneus in a medio-lateral direction; and
fixing the plate to the calcaneus.
